(19) United States
(12) Patent Application Publication
(10) Pub. No.: US 2015/0328032 A1
(43) Pub. Date: Nov. 19, 2015

(54) SMART KNEE FIXTURE AND SYSTEM FOR MEASURING KNEE BALANCING

(71) Applicants: Peter S. Walker, New York, NY (US); Ilya Borukhov, Rego Park, NY (US); Patrick A. Meere, Yorktown Heights, NY (US); Christopher Bell, Jersey City, NJ (US)

(72) Inventors: Peter S. Walker, New York, NY (US); Ilya Borukhov, Rego Park, NY (US); Patrick A. Meere, Yorktown Heights, NY (US); Christopher Bell, Jersey City, NJ (US)

(73) Assignee: New York University, New York, NY (US)

(21) Appl. No.: 14/710,508

(22) Filed: May 12, 2015

Related U.S. Application Data
(60) Provisional application No. 61/992,332, filed on May 13, 2014.

(51) Int. Cl. A61F 5/01; A61F 5/30

(52) U.S. Cl. CPC A61F 5/0109; A61F 5/30

(57) ABSTRACT

A Smart Knee Fixture (SKF) is a system comprising sensors, exciters and computation and command controllers that instrument the characterization of anatomical knee balance. Specifically the instrumentation allows the measurement of the relationship between varus-valgus angular response and a measured side force moment.

### PRIORITY CLAIM TO PREVIOUS PATENT APPLICATION

This U.S. utility patent application claims the benefit of U.S. Provisional Patent Application No. 61/992,332 that was filed on 13 May 2014, the entire disclosure of which is incorporated by reference in its entirety. All references cited in this specification, and their references, are incorporated by reference herein where appropriate for teachings of additional or alternative details, features, and/or technical background.

### BACKGROUND

Knee braces of numerous design have long been used for the stabilization of the knee, typically after soft tissue injuries and after reconstructive surgery. Braces have also been used prophylactically to prevent injuries in certain sporting activities. However it is only in recent years that braces have been instrumented to measure different functional parameters. Kobashi (2008), Atallah (2011), Shull (2013), Senanyake (2013) described wearable sensors, in some cases using wireless technology, for gait analysis and angular measurement monitoring, and retraining after injury or surgical procedures. Wang (2011) and Toffola (2012) constructed an electro-goniometer to measuring the flexion angle of joints using flexible polymeric material whose resistance changed with length. Khan (2012) measured knee stability after total knee replacement using an accelerometer, and identified many patients with instability that could be associated with imbalance. The measurement of anterior-posterior stability using the KT 1000/2000 devices is now standard practice in the sports medicine area. The applicants have identified the need for a device having the capability to measure equally important varus-valgus stability.

### BRIEF SUMMARY

The proper function of the knee whether in daily activities, sports, or after a knee replacement, is critically dependent on the complex interaction and balance of the numerous ligaments that surround the joint. In embodiments there is disclosed an apparatus, or Smart Knee Fixture (SKF), that answers the need for a device with the capability of measuring the kinematic function of the knee in general and varus-valgus stability, in particular. The SKF may comprise a combination of intelligent stretchable sensors that measure linear displacement, accelerometers or acoustic sensors, and an acoustic generator device mounted on a neoprene-type knee fixture. The SKF provides the capability to measure and characterize the kinematic function of the knee while not hindering its normal function.

In an embodiment, the SKF comprises a fixture that attaches across the knee joint and enables the measurement of varus and valgus laxity, in order to quantify knee balance. The device is designed primarily for the assessment of patients after injury, and before and after procedures including ligament or meniscal repairs, and knee replacement.

In use, equal varus and valgus moments of force are externally applied by applying forces at the malleoli at the ankle. The forces are measured by a load-sensing device, and recorded on a computer, in the varus and valgus directions without overstressing the knee and causing pain. In an embodiment, the SKF employs a force gage, positioned at the ankle, in combination with instrumentation that allows the determination of the point of lift-off of one of the condyles when the force moment is applied. A varus moment will cause lift-off of the lateral condyle while a valgus moment will cause lift-off of the medial condyle. The ankle moments on varus and valgus lift-off are the quantities used for defining the balancing. Equal moments indicate a perfectly balanced knee.

The instrumentation for determination of lift-off may be based on the detection of an acoustic signal across the femoral-tibial condylar interface. An acoustic wave at, for example, an ultrasonic frequency, may be coupled via a transducer into the tibia below the knee joint. The conducted acoustic wave may be separately sensed on the medial and lateral sides of the femur above the knee. The lift-off of the medial or lateral condyles may be determined by a change in the detected wave as sensed by the medial or lateral sensors respectively.

The angles of varus or valgus deviation may be are measured with stretch sensors mounted at the lateral and medial sides of the knee. The stretch sensors generate an electrical signal or change an electrical property indicating the linear dimension between two points to which the sensor is affixed. The mountings may be shaped for anatomical fit and may be fabricated using 3-D printing technology. The mountings include a means for length adjustment so that the stretch sensors are at an appropriate initial tension with the knee at or near extension.

The concept has been validated using cadaver studies to determine the relation between the electric resistance of the stretch sensors with the varus or valgus angles. In addition validation was performed using fluoroscopic images to quantify angles and the lift-off on the lateral and medial sides. During this validation electromyography was used to demonstrate no muscle activity within the limits of our testing procedure.

### FIGURES

The disclosure may be aided by the figures of an embodiment of the SKF as follows:

FIG. 1 is a portrayal of the operation of an SKF.

FIG. 2 is a portrayal of an exemplary configuration of an SKF.

FIG. 3 is a functional block diagram of an SKF.

### DETAILED DESCRIPTION OF EMBODIMENTS OF THE INVENTION

The Smart Knee Fixture (SKF) may comprise a tibial vibration source that can generate an acoustical signal, one or more acoustic sensors that are sensitive to the acoustic signal, one or more stretch sensors that measure the distance across the gap between a condyle and its corresponding tibial surface, and the electronics and computational equipment necessary to support the SKF. Wired or wireless connection may be employed between the various components.

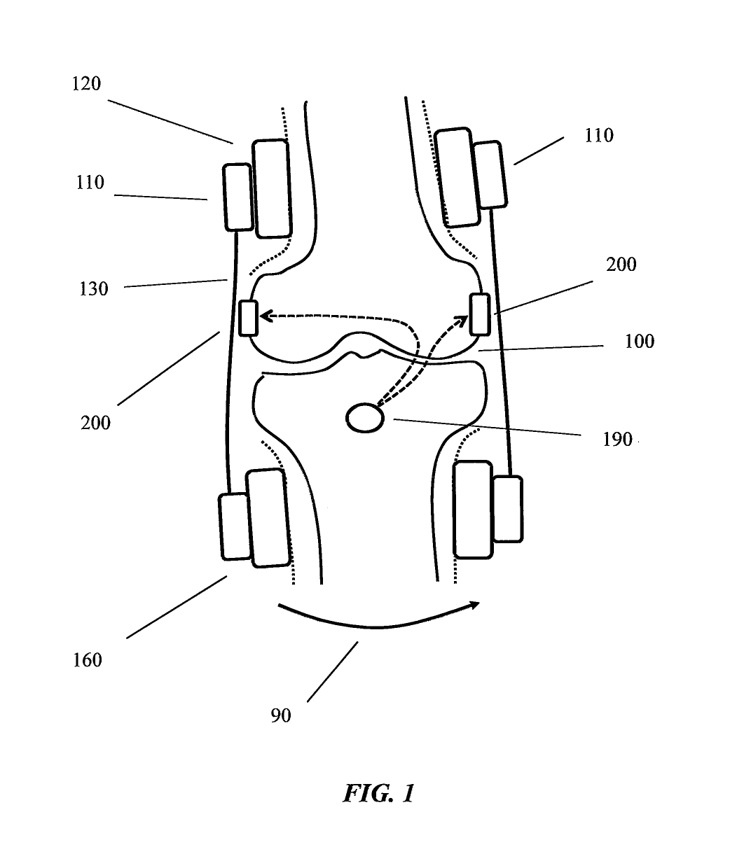

As portrayed in FIG. 1, a Smart Knee Fixture (SKF) provides the ability to measure the mechanical moment at which lift-off of a condyle from the tibia occurs. A source of acoustic vibration 190 is held in contact with the surface of the leg thereby causing vibrational energy to be mechanically coupled into the tibia. Initially there is contact on both pairs of condyles (one pair=femoral+tibial condyles). The vibration from the tibia goes through both pairs of condyles. A sideward force 90 is gradually applied to the leg near the foot causing a varus moment to be applied to the tibia. When lift-off of the lateral side occurs, the vibration path on the lateral side is interrupted and the vibration passes only thru the medial condyle. This sudden change is detectable by the relative responses of the two acoustic sensors 200 positioned on the lateral and medial sides of the femoral condyles respectively. It is noted that, in an alternate embodiment, a lone acoustic sensor positioned on the femur can be used to detect the difference in the vibrational signal as lift-off occurs.

Similarly, a valgus mechanical moment may be applied to the tibia. In this case, the medial condyle will lift-off resulting in the interruption of the vibration path on the medial condyle. The varus and valgus moments required to cause lateral and medial condyle lift-off, respectively, can be determined and compared.

Stretch sensors 130 may be positioned on the lateral and medial sides of the knee to measure the change in condylar gap distances. These measurements may be employed to compute the angles of varus and valgus deformity and, in addition, be used to measure additional varus or valgus angles after lift-off has occurred.

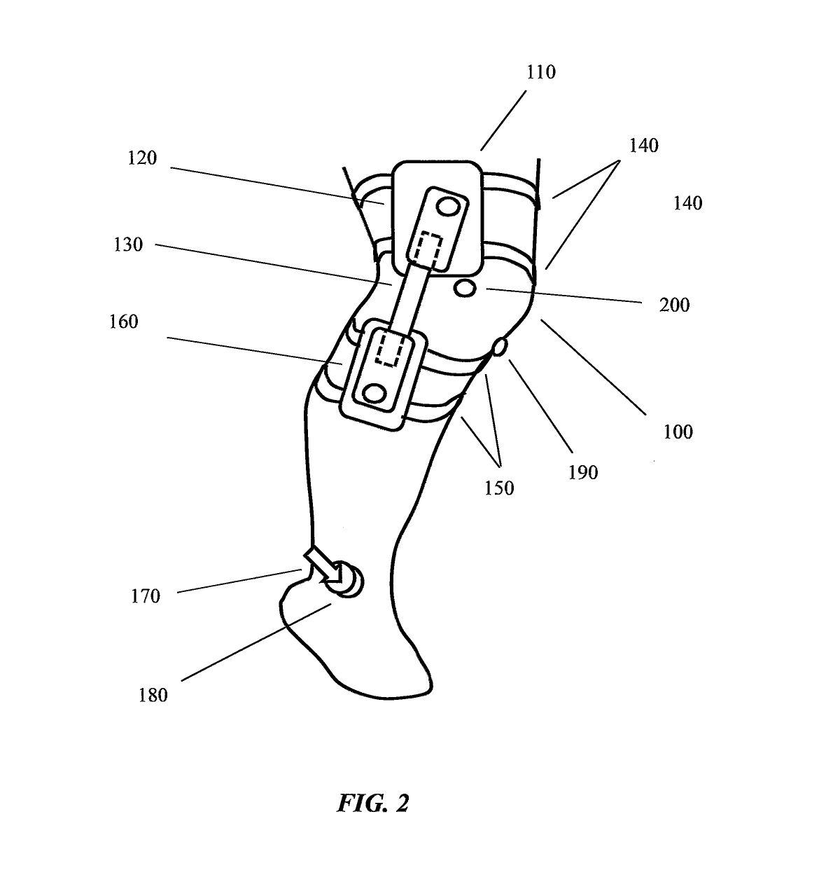

As shown in FIG. 2, the Smart Knee Fixture (SKF) may be mounted around the knee joint 100 and comprises femoral parts and tibial parts. The femoral parts include two femoral fixtures 110 that attach against the lateral and medial sides of the leg. Each fixture has an adjustable block 120 that allows for variation of length and orientation of a stretch sensor 130. The adjustable block 120 can also allow for correct orientation of the sensors 130 during dynamic activities where the angle of flexion of the knee joint 100 is continuously changing. The fixtures 110 are attached to the thigh by two femoral straps 140. Likewise two tibial straps 150 attach tibial fixtures 160 to the lower leg. The upper ends of the stretch sensors 130 are attached to the femoral fixtures 110 while the lower ends of the stretch sensors 130 are attached to the tibial fixtures 160. After mounting on the leg, the positions of the sensors are measured to allow the trigonometric calculation of the varus and valgus angles.

In order to apply a varus or valgus moment to the knee, a sideways force 170 is applied at the ankle using a handheld dynamometer 180, as shown in FIG. 2. The handheld dynamometer 180 may be wireless. The magnitude of the applied moment is equal to the applied force times the length of the lower leg from the ankle to the knee joint at the level of the joint line. The angular deviation between the thigh and the lower leg is measured by the stretch sensors 130 which have been previously been calibrated.

In order to detect the point of condylar lift-off when a moment is applied, an acoustic vibrator 190 and acoustic sensors) 200 may be used. A high frequency vibration, generated by the acoustic vibrator 190 may be applied to the tibial tubercle. The vibration motor may be operated by a low voltage (i.e., 5 volts for example), typically produces a vibration of approximately 6 G. The vibration travels through the knee joint and is detected at the lateral and medial femoral condyle by the acoustic sensors 200. The respective detected signals are characterized by two separate responses due to different pathways through the lateral and medial condyle contacts. This detected signal changes to a single response when one condyle lifts off. As the mechanical moment is applied at the ankle, at the point of lift-off, the value of the applied moment and the angular deviation are taken as the measures for either varus or valgus. The comparison of the varus and valgus values indicate the balancing, equal balancing being indicated by equal varus and valgus values.

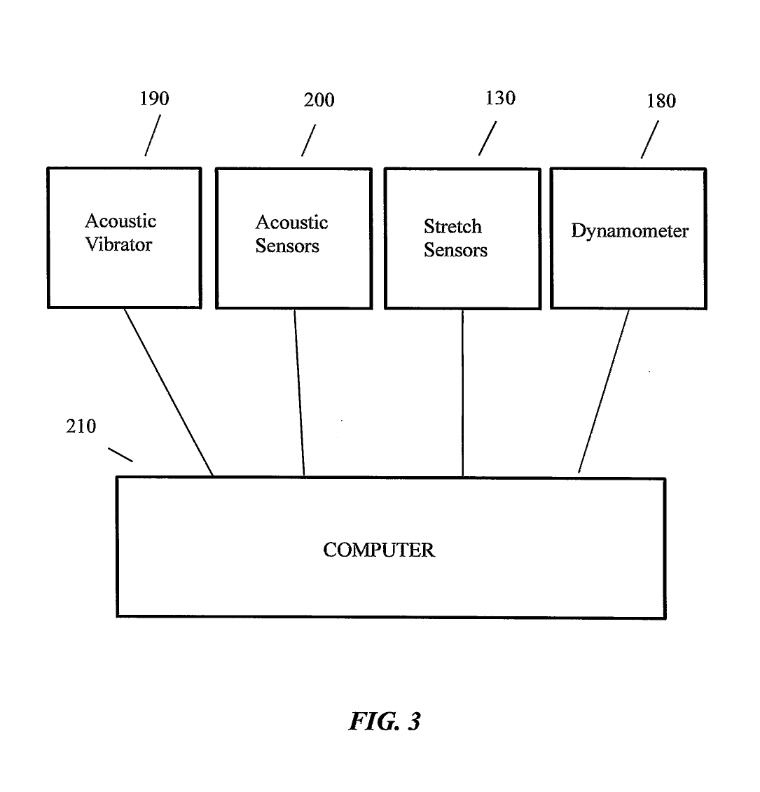

An embodiment of an SKF system is presented in FIG. 3. The acoustic vibrator 190, acoustic sensors 200, stretch sensors 130, and dynamometer 180 units are functionally connected to the computer 210. Alternative embodiments my comprise less than the full complement of vibrators and sensors. The computer 210 may be a centralized unit or, alternatively, may be distributed amongst the component units. The interconnection of the units may be wired or wireless. The computer 210 may be a general purpose or special purpose computer that provides the commands and signal required to operate the connected units and also receives the data from the sensors and dynamometer. The computer 210 may generate the commands and/or signals necessary to the operation of the connected units. The data received from the sensors and dynamometer may be processed, analyzed and logged by the computer 210. The computer may provide the results of the computation in any GIF or print format.

In the case of measurements with total knee replacement, the bearing surfaces are sufficiently rigid that on application of a varus or valgus moment, at a certain moment, lift-off will start to occur, before which there will be no detected changes in angulation. Hence, an alternate way of detecting lift-off is the force applied by the force gage (i.e., dynamometer 180) at the ankle is continuously monitored along with angulation, such that at the point of lift-off, the moment to cause lift-off will be determined, and then the subsequent angulation as well as the increased moment will be measured.

While the fixture has an application for the measurement of varus and valgus moments at a fixed angle of flexion such as 10-20 degrees, by virtue of a swivel connection of the adjustable block 120 the fixture can be used in a dynamic activity to measure condylar lift-off and angular deviations in dynamic activities, even in active sports. It will be recognized that the construction of the fixture can vary while still maintaining the principles of operation.

Applications of the device include monitoring the coronal plane stability after total knee replacement or other surgeries, and improving surgical techniques.

Other potential applications include the early detection of potential ligamentous injury associated with sporting activities, one example being multiple ligamentous knee injuries in teenage female soccer players. In this case, the SKF may be used in a dynamic mode to detect episodes of condylar lift-off or excess varus and valgus angular deviations.

Statement Regarding Preferred Embodiments

While the invention has been described with respect to the foregoing, those skilled in the art will readily appreciate that various changes and/or modifications can be made to the invention without departing from the spirit or scope of the invention defined by the appended claims.

What we claim is:

1. An apparatus comprising:
a femoral fixture comprising a medial femoral pad and a lateral femoral pad adjustably secured by at least one strap configured to wrap around a femoral portion of a leg;
a tibial fixture comprising a medial tibial pad and a lateral tibial pad adjustably secured by at least one strap configured to wrap around a tibial portion of a leg;
an elongated medial stretch sensor having a first end and a second end, said medial stretch sensor produces an output signal, at an input/output connection, proportional to the distance between said first end and said second end, said first end of said medial stretch sensor physically connected to said medial femoral pad and said second end physically connected to said medial tibial pad;
an elongated lateral stretch sensor having a first end and a second end, said lateral stretch sensor produces an output signal, at an input/output connection, proportional to the distance between said first end and said second end, said first end of said lateral stretch sensor physically connected to said lateral femoral pad and said second end physically connected to said lateral tibial pad;
a dynamometer that produces an output signal, at an input/output connection, proportional to the external force applied to said dynamometer; and
a computer comprising a processor, a memory, in input/output interface that is operatively connected to said input/output connection of said medial stretch sensor, said input/output connection of said lateral stretch sensor, and said input/output connection of said dynamometer.

2. An apparatus, in accordance with claim 1, further comprising:
an acoustic vibrator that can produce a high frequency mechanical vibration, said acoustic vibrator is controlled by said computer via an input/output connection and is operatively configured to fasten to the front tibial surface;
a first acoustic sensor that produces an output signal, at an input/output connection, proportional to said high frequency mechanical vibration and is operatively configured to fasten to the medial femoral surface;
a second acoustic sensor that produces an output signal, at an input/output connection, proportional to said high frequency mechanical vibration and is operatively configured to fasten to the lateral femoral surface;
wherein said computer is further connected to said input/output connections of said first and second acoustic sensors and to said input/output connection of said acoustic vibrator, said computer further can generate a control signal for said acoustic vibrator.
